(19) United States
(12) Patent Application Publication
(10) Pub. No.: US 2005/0186275 A1
(43) Pub. Date: Aug. 25, 2005

(54) Sustained release tamsulosin formulations

(75) Inventors: Hsiao Ching-fen, Chiayi (TW); Wang Yi Loong, Kaohsiung Hsien (TW); Ya Sheng Yang, Hsinying City (TW); Yaching Changchien, Kaohsiung (TW)

Correspondence Address: Cooper & Dunham LLP, 1185 Avenue of the Americas, New York, NY, 10036, US

(73) Assignee: Standard Chem. & Pharm. Co. Ltd.

(21) Appl. No.: 10/784,499

(22) Filed: Feb. 23, 2004

(51) Int. Cl. A61K 9/22; A61K 9/26

(52) U.S. Cl. 424/468

(57) ABSTRACT

A sustained release tamsulosin formulation contains tamsulosin, a hydrophobic polymer, a microsphere forming agent and a diluent. The hydrophobic polymers include pH-dependent and pH-independent polymers are used as the release-modulating agent to control the dissolution profile of tamsulosin formulation so that the formulation releases tamsulosin slowly and continuously as the formulation passed through the stomach and gastrointestinal tract.

### BACKGROUND OF THE INVENTION

1. Field of Invention

The present invention relates to a sustained release tamsulosin formulation.

2. Description of the Related Art

The mechanism of physiological action of tamsulosin is through blocking the -receptors actions in the cells of the urethra and prostate, so that the stress of a prostate is reduced and the difficulties with the flow of urine due to hypertrophy of the prostate are alleviated.

U.S. Pat. No. 4,772,475 discloses a formulation including controlled release formulations comprising an acrylic acid polymer, an acrylic acid copolymer or a cellulose derivative. More than 50% (w/w) microcrystalline cellulose was added into an oral sustained release formulation as a release-modulating agent. However, the high concentration of microcrystalline cellulose would increase the friction when a formulation mixture is kneaded and also elevate the temperature of the formulation mixture during the process of granulation. Also, acrylic acid polymer becomes glue-like under a high temperature, and the glue-like status of acrylic acid polymers in granulation procedures is unfavorable.

There is still a need in the related art to provide a sustained release of tamsulosin formulation, which can overcome the problems of resulted from temperature increase the glue-like status of acrylic acid polymer in the process of granulation, and maintain the desired extended-release effect.

### SUMMARY OF THE INVENTION

An aspect of the present invention is to provide a sustained release pharmaceutical composition of tamsulosin.

The pharmaceutical composition of the present invention contains tamsulosin or a pharmaceutical acceptable salt thereof, a hydrophobic polymer, a microsphere forming agent, and a diluent.

In a preferred embodiment of the present invention, the pharmaceutical composition contains tamsulosin or a pharmaceutical acceptable salt thereof, a hydrophobic polymer in a range from about 10% to about 65% (w/w), a microsphere forming agent in a range from about 20% to about 65% (w/w), and a diluent in a range from about 10% to about 40% (w/w).

Other aspects, advantages and novel features of the invention will become more apparent from the following detailed description when taken in conjunction with the accompanying drawings.

### BRIEF DESCRIPTION OF THE DRAWINGS

FIG. 1 shows the bio-equivalence comparison of a conventional sustained release tamsulosin formulation and a sustained release tamsulosin formulation of the present invention;

FIG. 2 shows the dissolution profiles of a sustained release tamsulosin formulation of the present invention (test) and a conventional tamsolusin formulation (reference).

### DETAILED DESCRIPTION OF THE INVENTION

In accordance with the present invention, a sustained release tamsulosin formulation is provided.

The term “sustained release” as used herein refers to formulation or dosage units of this invention that are slowly and continuously dissolved and absorbed in the stomach and gastrointestinal tract over a period of time.

The term “microsphere forming agent” as used herein refers to any binders of this invention that are used for forming a granule. The microsphere forming agent for the present invention also uses for making the size of the granulation equalized.

The term “fluctuation” as used herein refers to the changes of the concentration of tamsulosin in a human body. A smaller fluctuation of the sustained release tamsulosin formulation refers to the concentration of tamsulosin in blood is more stable.

The term “procedures” as used herein refers to the way to mix compounds.

The term “steps” as used herein refers to the order of producing the sustained release tamsulosin formulation.

Polymers have been widely used as a matrix for sustained release formulations. Hydrophobic polymers are suitably employed in the sustained release formulation, including pH-dependent and pH-independent polymers.

To provide for a sustained release tamsulosin formulation of the present invention, hydryphobic polymers (include pH-dependent and pH-independent polymers) are chosen to control the dissolution profile of tamsilosin formulation so that the formulation releases tamsulosin slowly and continuously as the formulation passed through the stomach and gastrointestinal tract. The dissolution control capacity of such polymers is particularly important in a sustained release tamsulosin formulation because it may cause damping effects if the tamsulosin is released too rapidly.

The hydrophobic polymers used for the invention are those which could avoid burst out of drug during its residence in the stomach and gastrointestinal tract. Many materials known as the hydrophobic polymers in the pharmaceutical art include sodium carboxymethyl cellulose, cellulose acetate, ethyl cellulose (EC), hydroxypropyl methyl-cellulose acetate succinate (HPMCAS) and cellulose acetate propionate (CAP). These polymers may be used alone or mixed in a sustained release tamsulosin formulation. The pH-dependent polymers are present in the tamsulosin formulation of this invention in an amount ranging from about 10 to about 65 wt %.

A water-soluble tamsulosin, a microsphere forming agent, a release-modulating agent and a diluent are mixed to obtain a mixture. Preferred microsphere forming agents that can be used in forming granules containing tamsulosin are glyceride or wax. Preferred release-modulating agents that can be used for delaying release rate of tamsulosin are the hydrophobic polymers. The mixture was added into a knead solution to form an oral dosage unit. An oral dosage unit of the sustained release tamsulosin formulations of this invention may be in the form of a capsule or a granule. The granules may be coated one or more films for different purposes and then encapsulated.

In preferred embodiments of the present invention, the range of the concentration of tamsulosin in the sustained release tamsulosin formulations is from about 0.03% to about 3% by weight based on the weight of the oral dosage unit (w/w). The range of the concentration of the hydrophobic polymers in the sustained release tamsulosin formulations is about 10% to about 65% by weight based on the weight of the oral dosage unit (w/w). The hydrophobic polymers are used to lower the fluctuation of the formulation in a human body. The range of the concentration of the microsphere forming agent in the sustained release tamsulosin formulations is about 20% to about 65% by weight based on the weight of the oral dosage unit (w/w). The microsphere forming agent also has the lubricating effect and can make the procedure of granulation favorable. Also, the glue-like status of the acrylic polyol polymers under a high temperature can be improved by the use of microsphere forming agent.

The sustained release tamsulosin formulation of the present invention may also contain pharmaceutical diluent. The range of the concentration of the diluent in the sustained release tamsulosin formulation of the present invention is about 10% to about 40% by weight based on the weight of the oral dosage unit (w/w).

The preferred diluents according to the present invention could be lactose, starch, mannitol, sodium hydroxylpropyl cellulose, sodium starch, microcrystalline cellulose, glyceryl behenate, talcum powder, stearic acid, stearic salt and sodium stearyl fumarate.

The preferred microsphere forming agents according to the present invention contain glyceryl triacetate, glyceryl monostearate, glyceryl behenate, paraffin wax and carnauba wax.

A method of conducting film coating is described as follows. A film coating premix is solved in water and organic solvent. The organic solvent used for preparing the film coating premix could be alcohol, acetone or isopropanol. Other diluents or anti-adhesive agents can be added into the above solvent mixture if necessary.

Other features and advantages of the present invention will be apparent from the following description of the preferred embodiments and from the claims.

### EXAMPLES

The following examples illustrate various aspects of the present invention but do not limit the claims in any manner whatsoever.

### Example 1

### Sustained Release Tamsulosin Formulation (1) and Method for the Production Thereof

Sustained Release Tamsulosin Formulation (1)

| | | |
|---|---|---|
| (a) | tamsulosin HCl | 1.00 g |
| | microcrystalline cellulose (MC) | 208.5 g |
| | stearic acid | 58 g |
| | glyceryl behenate | 290 g |
| | methacrylic copolymer | 22.5 g |
| (b) | film coat | |
| | sodium carboxymethyl cellulose | 0.85 g |
| | talcum powder | 27 gw |
| | triethyl citrate | 10.5 g |
| | methacrylic copolymer | 105.5 g |

Procedures:

The sustained release tamsulosin formulations of this invention are prepared as follows:

1. Tamsulosin HCl, microcrystalline cellulose, stearic acid and glyceryl behenate are intimately mixed.

2. Methacrylic copolymer was wet-blended.

3. Film coat premix 1: sodium hydroxylpropyl cellulose, methacrylic copolymer, talcum powder and triethyl citrate were mixed well.

Steps:

1. The mixture obtained from Example 1 Procedure 1 was mixed with the mixture obtained from Example 1 Procedure 2.

2. The mixture obtained from Step 1 was put into an extruding granulator and centrifugal spheroider to form a granule.

3. The granules were dried in a tray dryer.

4. The dried granule was put into a fluidized bed coater, and the film coat premix 1 solved in the selected solvent was sprayed on an outer surface of the granule.

### Example 2

### Sustained Release Tamsulosin Formulation (2) and Method for the Production Thereof

Sustained Release Tamsulosin Formulation (2)

| | |
|---|---|
| ethyl cellulose (EC) | 134 g |
| hydroxylpropyl cellulose | 0.6 g |
| talcum powder | 10.8 g |
| triethyl citrate | 8.4 g |

Procedures:

Film coat premix 2: ethyl cellulose, hydroxypropylmethyl cellulose (HPMC), talcum powder and triethyl citrate were mixed well.

Steps:

1. The granules obtained from Example 1 were put into a fluidized bed coater, and the film coat premix 2 solved in a selected solvent was sprayed on the above granules.

### Example 3

### Sustained Release Tamsulosin Formulation (3) and Method for the Production Thereof

Sustained Release Tamsulosin Formulation (3)

| | | |
|---|---|---|
| (a) | tamsulosin HCl | 0.40 g |
| | microcrystalline cellulose (MC) | 54.0 g |
| | hydroxypropyl methyl cellulose succinate | 20.0 g |
| | (HPMCAS) | |
| | stearic salt | 2.0 g |
| | glyceryl behenate | 134.0 g |
| | ethyl cellulose | 98.6 g |

Procedures:

1. Tamsulosin HCl, microcrystalline cellulose, hydroxypropyl methyl cellulose acetate succinate (HPMCAS), stearic salt and glyceryl behenate were mixed well.

2. Ethyl cellulose was wet-blended.

Steps:

1. The mixture obtained from Example 3 Procedure 1 was mixed with the mixture obtained from Example 3 Procedure 2.

2. The mixture obtained from Step 1 was put into an extruding granulator and centrifugal spheroider to form a granule.

### Example 4

### Sustained Release Tamsulosin Formulation (4) and Method for the Production Thereof

Sustained Release Tamsulosin Formulation (4)

| | | |
|---|---|---|
| (a) | tamsulosin HCl | 0.40 g |
| | microcrystalline cellulose (MC) | 39.2 g |
| | hydroxypropyl methyl cellulose | 20.0 g |
| | acetate succinate (HPMCAS) | |
| | stearic salt | 6.4 g |
| | glyceryl behenate | 120.0 g |
| | ethyl cellulose | 113.3 g |

Procedures:

1. Tamsulosin HCl, microcrystalline cellulose, hydroxypropyl methyl cellulose acetate succinate (HPMCAS), stearic salt and glyceryl behenate were mixed well.

2. Ethyl cellulose was wet-blended.

Steps:

1. The mixture obtained from Example 4 Procedure 1 was mixed with the mixture obtained from Example 4 Procedure 2.

2. The mixture obtained from Step 1 was put into an extruding granulator and centrifugal spheroider to form a granule.

### Example 5

### Sustained Release Tamsulosin Formulation (5) and Method for the Production Thereof

Sustained Release Tamsulosin Formulation (5)

| | | |
|---|---|---|
| (a) | tamsulosin HCl | 0.40 g |
| | microcrystalline cellulose (MC) | 39.2 g |
| | cellulose acetate phthalate (CAP) | 20.0 g |
| | magnesium stearate | 6.4 g |
| | glyceryl behenate | 120.0 g |
| | ethyl cellulose | 113.3 g |

Procedures:

1. Tamsulosin HCl, microcrystalline cellulose, cellulose acetate phthalate (CAP), sterate and glyceryl behenate were mixed well.

2. Ethyl cellulose was wet-blended.

Steps:

1. The mixture obtained from Example 5 Procedure 1 was mixed with the mixture obtained from Example 5 Procedure 2.

2. The mixture obtained from Step 1 was put into an extruding granulator and centrifugal spheroider to form agranule.

### Example 6

### Sustained Release Tamsulosin Formulation (6) and Method for the Production Thereof

Sustained Release Tamsulosin Formulation (6)

| | | |
|---|---|---|
| (a) | microcrystalline cellulose (MC) | 39.2 g |
| | cellulose acetate phthalate (CAP) | 20.0 g |
| | sterate | 6.4 g |
| | glyceryl behenate | 66.0 g |
| | ethyl cellulose | 50.0 g |
| (b) | castor oil | 20.0 g |
| | tamsulosin HCl | 0.40 g |
| | ethyl cellulose aqueous dispersion | 126.67 g |

Procedures:

1. Microcrystalline cellulose, sterate, cellulose acetate phthalate (CAP), glyceryl behenate and ethyl cellulose were mixed well.

2. Tamsulosin HCl and ethyl cellulose aqueous dispersion were mixed well.

Steps:

1. The mixture obtained from Example 6 Procedure 1 was mixed with castor oil.

2. The above mixture obtained from Step 1 was mixed with Example 6 Procedure 2.

3. Then the mixture obtained from Example 6 Step 2 was put into an extruding granulator and centrifugal spheroider to form a granule.

### Example 7

### Sustained Release Tamsulosin Formulation (7) and Method for the Production Thereof

Sustained Release Tamsulosin Formulation (7)

| | | |
|---|---|---|
| (a) | microcrystalline cellulose (MC) | 39.2 g |
| | cellulose acetate phthalate (CAP) | 20.0 g |
| | magnesium stearate | 6.4 g |
| | glyceryl behenate | 66.0 g |
| | ethyl cellulose | 70.0 g |
| | tamsulosin HCl | 0.40 g |
| | ethyl cellulose aqueous dispersion | 133.33 g |

Procedures:

1. Microcrystalline cellulose, cellulose acetate phthalate (CAP), sterate, glyceryl behenate and ethyl cellulose were mixed well.

2. Tamsulosin HCl and ethyl cellulose aqueous dispersion were mixed well.

Steps:

1. The mixture obtained from Example 7, Procedure 1 was mixed with the mixture obtained from Example 7 Procedure 2.

2. Then the mixture obtained from Example 7 Step 2 was put into an extruding granulator and centrifugal spheroider to form a granule.

### Example 8

### Bio-equivalence

TABLE 1

| | | Sustained release |
|---|---|---|
| | conventional sustained | tamsulosin formulation |
| | release tamsulosin | from Example 5 of the |
| Parameters | formulation | present invention |
| [AUC]ss(ng/ml × hr) | 79.8 ± 33.5 | 82.8 ± 36.1 |
| fluctuation | 1.96 ± 0.576 | 1.79 ± 0.466 |
| Cₐᵥₑ^{ss} (ng/ml) | 3.32 ± 1.40 | 3.45 ± 1.50 |
| Cₘₐₓ^{ss} (ng/ml) | 7.72 ± 3.35 | 7.28 ± 2.70 |
| Cₘᵢₙ^{ss} (ng/ml) | 1.23 ± 0.651 | 1.42 ± 0.830 |
| Tₐᵥₑ^{ss} (hour) | 5.12 ± 1.31 | 3.92 ± 1.12 |

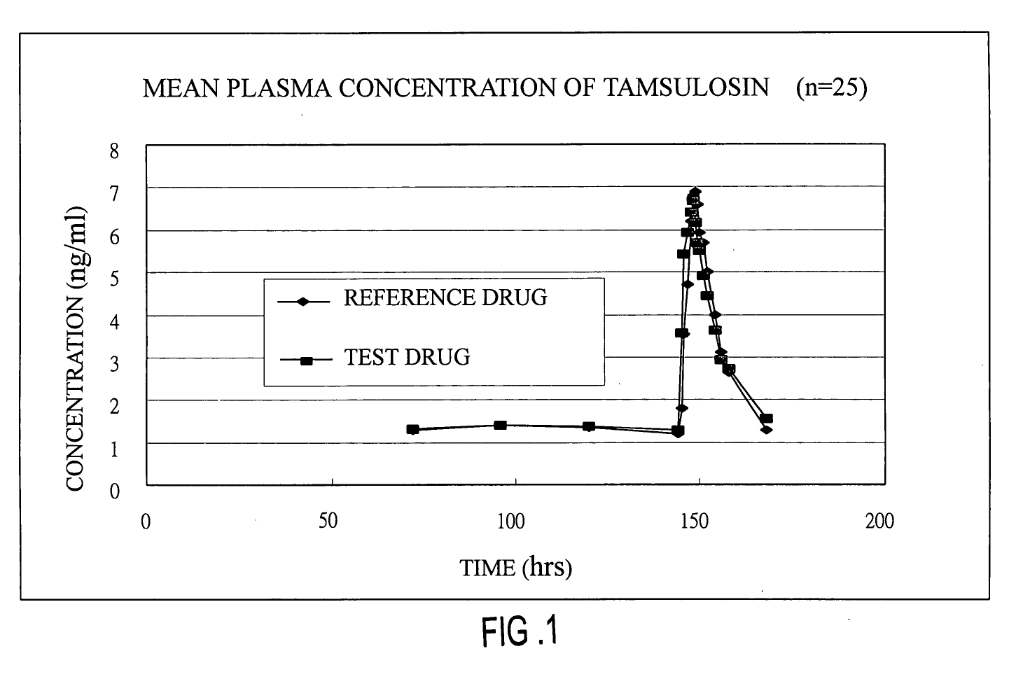

Tables 2 and 3 show the bio-equivalence of the conventional sustained release tamsulosin formulation and sustained release tamsulosin formulation of the present invention based on an analysis of the blood samples from 25 individuals. Results are shown in Table 2 and Table 3, and FIG. 1 depicts the comparison of the data given in Table 2 and Table 3.

TABLE 2
Time course of release of tamsulosin from a conventional sustained
release tamsulosin formulation

| | | Average | | |
|---|---|---|---|---|
| | | concentration | | |
| Time(hour) | Nos. | (ng/ml) | Standard Deviation | C.V.(%) |
| 72 | 25 | 1.28 | 0.858 | 67.1 |
| 96 | 25 | 1.42 | 0.974 | 68.6 |
| 120 | 25 | 1.34 | 0.749 | 55.9 |
| 144 | 25 | 1.20 | 0.6698 | 58.1 |
| 145 | 25 | 1.80 | 0.958 | 53.3 |
| 146 | 25 | 3.53 | 1.99 | 56.4 |
| 147 | 25 | 4.71 | 2.39 | 50.7 |
| 148 | 25 | 6.19 | 3.39 | 54.8 |
| 148.5 | 25 | 6.80 | 3.18 | 46.8 |
| 149 | 25 | 6.89 | 2.97 | 43.1 |
| 149.5 | 25 | 6.58 | 2.80 | 42.6 |
| 150 | 25 | 5.92 | 2.60 | 43.9 |
| 151 | 25 | 5.69 | 2.25 | 39.5 |
| 152 | 25 | 5.00 | 1.89 | 37.7 |
| 154 | 25 | 3.99 | 1.67 | 41.7 |
| 156 | 25 | 3.11 | 1.50 | 48.2 |
| 158 | 25 | 2.65 | 1.31 | 49.4 |
| 168 | 25 | 1.30 | 0.781 | 60.2 |

TABLE 3
Time course of release of tamsulosin from an embodiment of the
sustained release tamsulosin formulation from Example 5
of the present invention

| | | Average | | |
|---|---|---|---|---|
| Time | | concentration | | |
| (hour) | Nos. | (ng/ml) | Standard Deviation | C.V.(%) |
| 72 | 25 | 1.32 | 0.797 | 60.2 |
| 96 | 25 | 1.40 | 0.661 | 47.4 |
| 120 | 25 | 1.38 | 0.608 | 44.1 |
| 144 | 25 | 1.28 | 0.759 | 59.5 |
| 145 | 25 | 3.57 | 1.63 | 45.6 |
| 146 | 25 | 5.41 | 2.58 | 47.7 |
| 147 | 25 | 5.92 | 2.36 | 39.8 |
| 148 | 25 | 6.42 | 2.59 | 40.3 |
| 148.5 | 25 | 6.67 | 2.78 | 41.6 |
| 149 | 25 | 6.18 | 2.38 | 38.5 |
| 149.5 | 25 | 5.68 | 2.15 | 37.8 |
| 150 | 25 | 5.50 | 2.01 | 36.5 |
| 151 | 25 | 4.92 | 2.03 | 41.2 |
| 152 | 25 | 4.43 | 2.09 | 47.2 |
| 154 | 25 | 3.63 | 1.83 | 50.4 |
| 156 | 25 | 2.94 | 1.46 | 49.8 |
| 158 | 25 | 2.72 | 1.48 | 54.2 |
| 168 | 25 | 1.57 | 0.996 | 63.3 |

The data in Table 2 and Table 3 show the fluctuations between conventional sustained release tamsulosin formulation and sustained release tamsulosin formulation obtained from the present invention. The sustained release tamsulosin formulation obtained from the present invention has a small fluctuation value, in other words, sustained release tamsulosin formulation obtained from the present invention is more suitable for patients than conventional tamsulosin form.

### Example 9

### Ingredient-releasing Rate Test

Experiments: Testing of ingredient-releasing rates of the tamsulosin medicine:

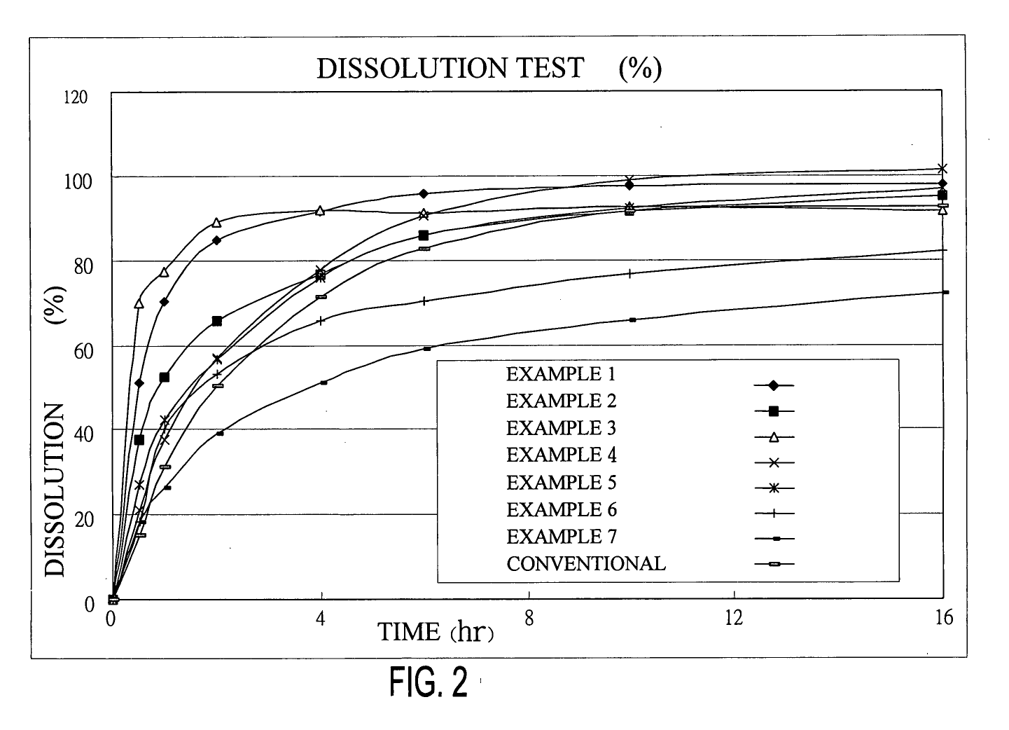

The rates of tamsulosin release from the tamsulosin formulations made according to Examples 1-7 (test) and a conventional sustained release tamsulosin formulation (reference) were evaluated in a dissolution test under the instructions of the United States Pharmacopoeia (U.S.P.)26^{th} edition. In this test, each tamsulosin formulation and 500 ml of pH 6.8 phosphate buffer were poured into a vessel and heated up to 37±0.5° C. Then, the mixture was paddled in a mixer at 100 rpm. The results are shown in Table 4 and FIG. 2.

TABLE 4
Ratios (%) of the tamsulosin released from the
formulations in pH 6.8 hydrochloric acid solution.

| Time | Example | Example | Example | Example | Example | Example | Example | |
|---|---|---|---|---|---|---|---|---|
| (hours) | 1 | 2 | 3 | 4 | 5 | 6 | 7 | Conventional |
| 0.00 | 0.00 | 0.00 | 0.00 | 0.00 | 0.00 | 0.00 | 0.00 | 0.00 |
| 0.50 | 51.18 | 37.61 | 69.90 | 20.97 | 27.01 | 18.14 | 18.32 | 15.14 |
| 1.00 | 70.33 | 52.41 | 77.54 | 37.82 | 42.23 | 40.29 | 26.47 | 31.16 |
| 2.00 | 84.65 | 65.80 | 89.18 | 56.99 | 56.76 | 53.24 | 39.14 | 50.29 |
| 4.00 | 91.45 | 76.67 | 91.76 | 77.88 | 76.15 | 65.88 | 51.06 | 71.50 |
| 6.00 | 95.68 | 85.87 | 91.10 | 90.38 | 85.73 | 70.35 | 59.15 | 82.68 |
| 10.00 | 97.57 | 91.46 | 92.66 | 98.95 | 92.34 | 76.71 | 65.87 | 91.34 |
| 16.00 | 97.93 | 94.90 | 91.47 | 101.38 | 96.69 | 82.10 | 72.14 | 92.47 |

According to Table 4 in view of FIG. 2, all tamsulosin formulations tested showed equal quantity, including conventional sustained release tamsulosin formulation.

Accordingly, the foregoing examples illustrated the following advantages of the sustained release tamsulosin formulations of the present invention:

1. In the process present invention, the use of a microsphere forming agent as a lubricant was surprisingly found to solve the problem resulted from the glue-like status of an acrylic acid polymer or acrylic acid copolymer kneaded under a high temperature.

2. Sustained release tamsulosin formulations of the present invention was found surprisingly to exhibit prolonged ingredient-releasing efficacy. Patients can benefit from a reduction in the frequency of taking such formulations.

Various modifications and variations of the present invention will be recognized by those persons skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in the art, are intended to be within the scope of the following claims.

1. A sustained release tamsulosin formulation, comprising 0.03% to 3% by weight tamsulosin or a pharmaceutically acceptable salt thereof,
a hydrophobic polymer present at about 10% to 65% w/w of the formulation,
a microsphere formulating agent present at about 20% to 65% w/w of the formulation, and
a diluent present at about 10% to 40% w/w of the formulation.

2. The sustained release tamsulosin formulation as claimed in claim 1, wherein the diluent is selected from the group consisting of lactose, starch, mannitol, sodium hydroxylpropyl cellulose, sodium starch, microcrystalline cellulose, glyceryl behenate, talcum powder, stearic acid, sterate and sodium stearyl fumarate.

3. The sustained release tamsulosin formulation as claimed in claim 1, wherein the hydrophobic polymer is selected from a group of pH-dependent polymers and pH-independent polymers.

4. The sustained release tamsulosin formulation as claimed in claim 3, wherein the hydrophobic polymer is selected from the group consisting of sodium carboxymethyl cellulose, cellulose acetate, ethyl cellulose (EC), hydroxypropyl methyl-cellulose acetate succinate (HPMCAS) and cellulose acetate phthalate (CAP).

5. The sustained release tamsulosin formulation as claimed in claim 1, wherein the microsphere forming agent is selected from the group consisting of glyceryl triacetate, glyceryl monostearate, glyceryl behenate, paraffin wax and carnauba wax.
